(19) United States
(12) Patent Application Publication
(10) Pub. No.: US 2011/0124894 A1
(43) Pub. Date: May 26, 2011

(54) METHOD FOR OBTAINING LONG CHAIN ALIPHATIC ALCOHOLS AND FATTY ACIDS FROM SUGAR CANE MUD AND RELATED WAX ESTERS

(76) Inventor: Rafael Almagro, Miami, FL (US)

(21) Appl. No.: 12/625,797

(22) Filed: Nov. 25, 2009

(51) Int. Cl. C07C 29/147; C09K 3/00; C11B 1/00; C07J 1/00

(52) U.S. Cl. 552/526; 568/886; 252/182.12; 554/21

(57) ABSTRACT

Methods are provided for the extraction of wax esters from sugar cane mud, isolation of the wax, saponification of the wax, either after isolation or in an intermediate un-isolated form to provide higher primary aliphatic alcohols (HPAA's) and fatty acids having nutritional and medicinal uses. The solid waste resulting from the process can be disposed of or utilized as a soil substitute following an optional water wash. Additionally, methods are provided for providing modified forms of the sugar cane mud or cachaza having reduced volumes and/or water content that require less storage space and capable of being stored for long periods of time with little or no biological degradation.

### REFERENCE TO RELATED APPLICATION

This application claims the benefit of U.S. Provisional Application No. 60/811,616, filed Jun. 7, 2006, which is hereby incorporated by reference.

### BACKGROUND

Materials such as sugar cane mud and beeswax are rich sources of wax esters having useful and desirable properties. More recently saponification of the wax esters or wax has been shown to provide very long chain aliphatic alcohols and long chain fatty acids. Both the long chain aliphatic alcohols and the long chain fatty acids have nutritional and medicinal uses. For example, policosanol, a mixture of long chain aliphatic alcohols, has been shown to provide cholesterol lowering benefits similar to the better known statins; simvastatin and provastatin and has additional beneficial properties which include smooth muscle cell proliferation, platelet aggregation, and LDL peroxidation. See, Ioanna Gouni-Berthold, J D, and Heiner K. Berthold, M D, PhD; American Heart Journal, Volume 143, Number 2, February 2002. Similarly, U.S. Pat. Nos. 5,284,873, 5,502,045, 5,444,054, 4,505,933, and 4,687,783, report the use of long chain fatty acids to treat prostate affections, to reduce cholesterol levels, to treat ulcerative colitis, and to treat multiple sclerosis as well as other neurological and dermatological diseases.

Plant residues resulting from the processing of sugar cane have been shown to be a particularly rich source of wax esters or wax from which the long chain alcohols and fatty acids can be derived. The residues from processing sugar cane contain a variety of components that include, but are not limited to plant parts or fiber, resins, oils, lignin, and wax. Currently the long chain alcohols and fatty acids are derived by the saponification of isolated and purified sources of the wax. Isolation of the wax esters typically includes the extraction of the wax from a plant residue, beeswax or other wax containing material with a solvent for the wax having very limited water solubility, such as for example a hydrocarbon, separating the resulting solution from the plant solids and finally separating the wax from the extraction solvent. Because the extraction solvents employed are typically substantially insoluble in water, they cannot be easily removed from the waste solids and related insoluble components. As a result the waste solids containing substantial volumes of an organic solvent become a waste disposal problem or require an energy intensive step to remove the entrapped organic solvent prior to its disposal or other use. What is needed is a method for: (a) extracting and separating the wax from the mud or other wax source to provide a form of wax that can be directly saponified and providing a waste solid that can be disposed of or used without an expensive solvent recovery process; (b) saponifying the un-isolated wax to provide long chain aliphatic alcohols and the salts of fatty acids; and (c) isolating the long chain alcohols and fatty acids in a form suitable for nutritional and medicinal uses. Preferred embodiments of the current disclosure additionally provide methods for preparing intermediate sugar cane muds having reduced volumes that can be stored for long periods of time without decomposition or degradation. The preferred muds additionally have reduced water content and fewer large insoluble particles that do not significantly contribute to the wax yields. The method described herein addresses these needs.

### SUMMARY

The plant residue from processing sugar cane, from beeswax, and from many other plant materials are a sources for a number of important and valuable natural products. Because such products are generally components of complex mixtures, their separation, isolation and purification can present formidable challenges.

Described herein is a method for extracting natural waxes from plant sources such as the residue from the processing of sugar cane, saponifying those waxes to provide useful long chain aliphatic alcohols and long chain fatty acids, and isolating the alcohols and fatty acids to provide compositions having nutritional and medicinal uses. Similarly, the method can be interrupted to provide a wax product and the saponification portion of the overall process can be utilized to saponify waxes isolated by other methods. Finally, preferred embodiments of the method utilize a modified form of the plant residue that has been reduced in volume by a classification step and at least partially dried.

The higher primary aliphatic C₂₄-C₃₆ alcohols obtained from sugar cane wax are referred to herein as HPAA's. One embodiment of the current disclosure involves first selecting a filter cake mud having up to about 90% water, preferably from about 10% to about 70% water and more preferably from about 20% to about 40% water. The filter cake mud is extracted with a water miscible organic solvent to form a mixture containing the solid fiber and the wax dissolved in the water miscible solvent. The mixture is clarified to provide a solid including fiber and a solution of the wax, the solution of wax is combined with water and a saponification reagent to form a saponification medium and the wax is saponified. Any HPAA's formed in the saponification medium are isolated from the mixture.

Preferred extractions are carried out by combining the filter cake mud with a water-miscible solvent and heating the mixture to about 40° C. to about 120° C., preferably from about 50° C. to about 90° C., and more preferably from about 60° C. to about 80° C. to extract the wax into the water-miscible solvent. The resulting warm slurry is agitated and clarified by filtering, centrifuging or by other means to provide a solid residue and an extract in the form of a solution or dispersion of wax in the water-miscible solvent. The resulting solvent-wet residue can be washed further with water to remove the water-miscible solvent. The resulting water-wet residue is substantially free of the solvent and can be directly disposed of or used as a form of topsoil, with or without drying.

A preferred filter cake mud is selected from treated or modified filter cake muds having reduced volumes and/or lowered water contents. Larger solid particles typically contain lower recoverable amounts of wax and increased water contents reduce the efficiency of wax extraction and facilitate microbial degradation of the mud. Treated filter cake muds provide for more efficient extractions, require less storage space, and are more resistant to microbial degradation during long term storage. As a result, treated or modified muds can be worked year round in smaller and more economical plants rather than seasonally following sugar cane harvest.

Preferred forms of treated or modified filter cake mud can be prepared by mixing raw filter cake mud with water to form a first slurry containing water soluble and water insoluble components, classifying the first slurry by removing or isolating a portion of the water insoluble components unable to pass through an opening having a sized ranging from about 14 to about 22 mesh, and more preferably a size of about 18 mesh, to form a second slurry, similarly containing water soluble and water insoluble components; isolating the water insoluble components from the second slurry to form a wet solid; and drying the wet solid to a water content of less than about 90% to form the treated or modified filter cake mud. More preferred water contents are less than about 70%, still more preferred less than about 40% and most preferred ranging from about 20% to about 40%. A preferred method of drying the wet solid includes exposing the solid dispersed over a broad area to sunlight until a desired water content is obtained.

Aspects of the methods described above can also be utilized to obtain wax from a sugar cane mud for use as a wax and as a raw material for the production of HPAA's and fatty acids. The method for obtaining a wax from a filter cake mud includes selecting a filter cake mud having a water content of up to about 90% water; extracting the filter cake mud with a water miscible organic solvent to form a mixture containing the solid fiber and the wax dissolved in the water miscible solvent; clarifying the mixture to provide a solution of the wax in the water miscible solvent and the solid fiber; solidifying the wax to form a slurry; and isolating the wax from the slurry. Preferred filter cake muds are treated or modified filter cake muds having reduced water contents of less than about 70% water. More preferred treated or modified muds contain less than about 40% water and the most preferred treated or modified muds contain from about 20% to about 40% water. Preferred water miscible solvents include lower aliphatic alcohols, typically C₁ to C₅ alcohols, and the most preferred lower aliphatic alcohol is ethanol. Typical methods for isolating solids can be utilized in this process and include filtration and centrifugation methods.

Aspects of the methods described above can similarly be utilized to provide a source of wax for saponification to produce HPAA's and/or long chain fatty acids. The wax provided can be in an un-isolated crude form, an isolated crude form or an isolated and purified form. The saponification method includes forming a saponification medium containing the wax, a water-miscible solvent, and a saponification reagent; saponifying the wax in the saponification medium to produce long chain aliphatic alcohols and fatty acids; and isolating the HPAA's from the saponification medium, typically by filtering or centrifuging. Preferred water miscible solvents include lower aliphatic alcohols, particularly ethanol. Preferred saponification reagents include an inorganic base and a preferred inorganic base includes an inorganic base selected from the group consisting of sodium hydroxide, potassium hydroxide, and combinations thereof. After removal of the HPAA's from the saponification medium, an acid can be added to the filtrate to protonate and precipitate the fatty acids produced. Mineral acids are generally preferred for protonation of the fatty acid salts. The precipitated fatty acids can be isolated by filtration, centrifugation and the like.

### DRAWINGS

FIG. 1 illustrates a method for transforming a raw sugar cane mud or cachaza into a modified mud containing smaller particles and/or having a lower water content.

FIG. 2 illustrates a method for processing a sugar cane mud utilizing (a) a water-miscible solvent to extract the wax contained therein and (b) a saponifying agent in combination with the water-miscible solvent to transform the wax into HPAA's and fatty acids.

FIG. 3 illustrates a method for processing a sugar cane mud utilizing a water-miscible solvent to extract and isolate the wax contained therein.

FIG. 4 illustrates a method for saponifying an isolated sugar cane wax utilizing a water-miscible solvent and a saponifying agent to provide HPAA's and fatty acids.

### DESCRIPTION

For the purposes of promoting an understanding of the principles of this disclosure, references will now be made to several embodiments and specific language will be used to describe the same. It will nevertheless be understood that no limitation of the scope of the disclosure is thereby intended, such alterations and further modifications and applications of the principles of the disclosure as described herein being contemplated as would normally occur to one skilled in the art to which the disclosure relates.

One aspect of this disclosure relates to a novel process for isolating a wax or wax ester from the solid residue obtained from processing sugar cane referred to as filter cake, filter cake mud, and cachaza.

A further aspect of this disclosure relates to a process for the saponification of the wax to provide long chain aliphatic alcohols, also referred to as higher primary aliphatic alcohols (HPAA's) and fatty acids. The saponification reaction can begin with either an isolated or intermediate un-isolated form of the wax having a range of purities as disclosed below.

A still further aspect of the current disclosure provides for a method of treating the raw sugar cane mud to give a modified form of the mud having a reduced volume and/or a reduced water content. Forms of the modified mud having a reduced volume and reduced water content can be stored for extended periods of time in reduced storage space without biological degradation. Because of its storage stability and reduced volume, the modified mud can provide a generally year round source of raw material rather than only a seasonal source of the mud.

A still further aspect of this current disclosure relates to a process that yields a solid waste product that can be directly disposed of or be utilized as a soil substitute without the expense of drying the solid to remove a substantially water-insoluble organic solvent prior to the solid's disposal or use.

Processing sugar cane results in the formation of a solid residue or cachaza composed of fibrous materials and other water insoluble plant derived components. One component that has attracted significant attention is a wax that is a mixture of the esters of several long chain aliphatic alcohols and fatty acids. Early uses for the wax depended on the waxes physical properties. However, more recently the wax has been recognized as a source of important components having nutritional and medicinal value. Traditional methods of obtaining the wax from cachaza have involved extraction of sugar cane mud with a water immiscible solvent, such as a hydrocarbon. Water contained within the mud interferes with the extraction of water-containing mud using a water-immiscible solvent. Because large amounts of the water immiscible solvent is commonly trapped within the porous structure of the fibrous waste product, solvent recovery or drying has been necessary to avoid the loss of large volumes of solvent and to provide a solid waste product free of the water immiscible solvent that can be disposed of or used as a soil substitute.

One aspect of the current disclosure utilizes a water-miscible solvent to contact the cachaza (regardless of the water content of the cachaza), dissolve and extract the wax. After separation of the resulting solution or dispersion from the solids, the water-miscible solvent can be removed by distillation to provide, upon cooling, a solid wax suspended in water. Depending on the solvent selected, contacting the cachaza can be carried out at reduced or elevated temperatures. Elevated temperatures are generally preferred. The wax can be isolated from the aqueous slurry by filtering, centrifuging, and the like, and dried to provide wax in a solid form. Depending on the purity level needed, additional treatments may be employed to improve the quality of the wax. Suitable treatments include, but are not limited to, decolorizing a solution of the wax with carbon; digesting the solid with water, the water-miscible solvent or mixtures thereof; and washing the solid wax with additional water, the immiscible solvent or mixtures thereof. If, during the drying process the wax melts, it can be solidified by cooling and the resulting solid cake broken into manageable pieces. The manageable pieces can be ground and screened to provide a desired particle size. A solvent is considered a water-miscible solvent if a 1:10 mixture of water and the solvent based on volume provides a homogeneous composition.

The waste solids resulting from the separation typically contain both water and the water-miscible solvent. Further washing with water will substantially remove the water-miscible solvent to provide a water-wet solid waste that can be directly disposed of or utilized without further treatment as a soil substitute. The water-miscible solvent can be recovered from the wash liquids by distillation and reused in the current process.

A further aspect of this disclosure involves the saponification of the wax in the presence of a saponification reagent in the presence of a water-miscible solvent. Although not required, the saponification mixture generally contains some water from the mud, whether modified or not. Preferred water-miscible solvents include C₁ to about C₅ alcohols. During the late stage of saponification the water-miscible solvent is typically removed by distillation and is replaced with a similar volume of water. As a result, the water content of the saponification medium can vary during the saponification process, typically comprising primarily water miscible solvent in the early stages of saponification and becoming primarily water by the end of saponification. Preferred saponification reagents include inorganic bases, particularly metal hydroxides such as for example sodium and potassium hydroxide. The wax saponified can be an isolated and/or purified form of wax obtained by any procedure or an un-isolated intermediate form of the wax dissolved or dispersed in a water-miscible solvent of the type obtained in the procedure described above. The use of the water-miscible solvent allows for the solvents to be separated from the saponification medium by distillation and recycled, provided the solvent's boiling point is lower than water or the solvent forms an azeotrope with water. Following saponification, the HPAA's can be removed from the aqueous slurry by filtration, centrifugation, and the like and dried to provide solid product. Acidification of the aqueous filtrate causes the fatty acids to protonate and precipitate. The resulting insoluble fatty acids can be isolated by filtration, centrifugation or other methods known to those skilled in the art. Depending on the quality levels needed, the long chain aliphatic alcohols and fatty acids can be upgraded by decolorizing a solution of the acids prior to protonating, or digesting or washing the solid with the water-miscible solvent or aqueous mixtures thereof.

A still further aspect of this disclosure involves a method for producing a modified mud or cachaza from the raw processed cachaza. The modified mud or cachaza produced has a reduced volume and reduced water content compared to the raw cachaza initially produced. Because of its reduced water content and reduced volume, the modified mud can be stored in smaller storage facilities and/or for longer periods of time without biological degradation. According to the new process, the raw cachaza is initially classified by size through a process that removes particles having a size greater than a selected cutoff. Work completed at this time suggests that the raw cachaza can be advantageously classified by removing any particles unable to pass through openings ranging from about 14 to about 22 mesh and preferably through openings of approximately 18 mesh. Solids capable of passing through such openings are retained and the water content adjusted to provide a modified mud or cachaza. For modified mud or cachaza that will be stored for an extended period of time, further drying is preferred. Based on the work carried out at this time, a water content of from about 10% to about 70% is preferred for improved extractions whereas a water content of from about 20% to about 40% can be readily obtained and has provided modified mud or cachaza suitable for storage for extended periods of time. Therefore, at least partial drying of the initially classified mud or cachaza is generally preferred.

A more detailed disclosure follows and refers to the appended drawings. The illustrations that follow are related to specific embodiments of the present disclosure and are not intended to represent any limitations thereof.

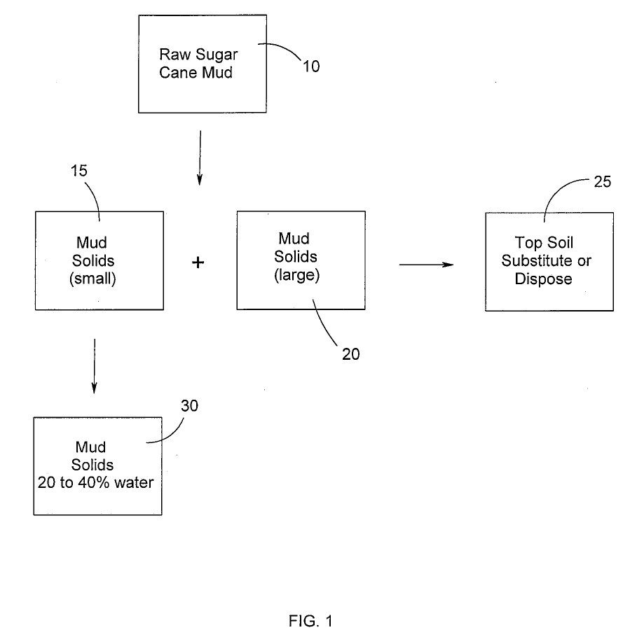

FIG. 1 illustrates the method for treating raw sugar cane mud or cachaza to form modified forms of the mud based on particle size and/or moisture content. A raw sugar cane mud 10 typical of the mud provided from sugar cane processing is classified by adding water, as necessary, to make the mixture fluid and then subjected to a surface having a plurality of openings of about 18 mesh. The solid particles that passed through the openings are collected to provide the modified mud 15. The solid particles that are unable to pass through the openings are collected to provide a second solid 20. Solid 20 can be disposed of without further treatment or utilized as a mulch or topsoil replacement. The modified mud solid 15, typically contains from about 70% to about 90% water and is suitable for further processing upon recovery. Modified solid 15 can also be further dried to provide preferred modified solid 30 containing from about 20% to about 40% water which is suitable for storing for future processing.

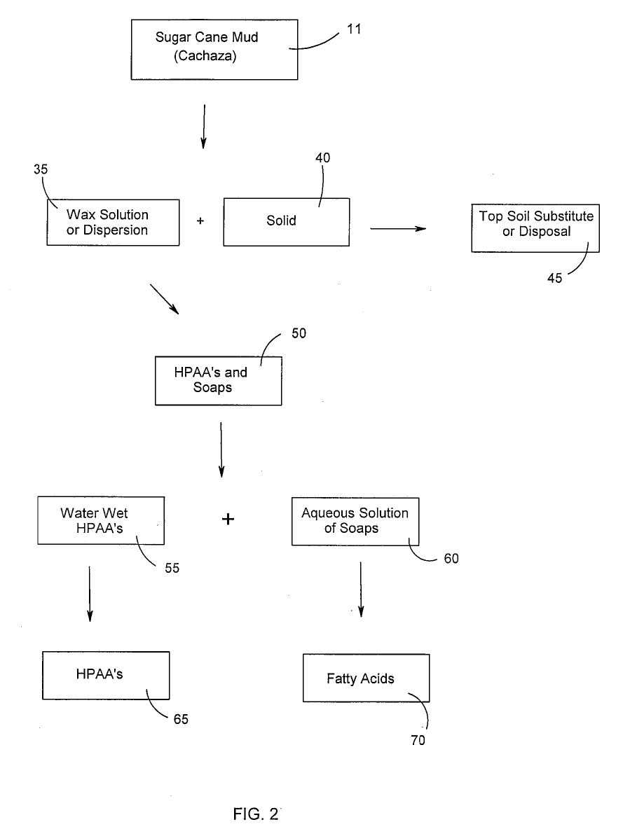

FIG. 2 illustrates a method for processing raw sugar cane mud or cachaza 11 to provide higher aliphatic alcohols 65 and fatty acids 70 useful for nutritional and medicinal purposes. Modified muds 15 and 30 can be advantageously substituted for the raw mud 11 in this process. Raw mud 11 is slurried with a sufficient amount of a water-miscible solvent to substantially dissolve and extract the wax contained in the sugar cane mud. Preferred water-miscible solvents include C-1 to C-5 alcohols, particularly ethanol. Preferred extractions are carried out at an elevated temperature to facilitate the extraction and increase the solubility of the wax in the water-miscible solvent. For water-miscible solvents that form a water azeotrope, it may be advantageous to reduce the water content of the mixture by removing a portion of the water as the azeotrope before separating the solids from the extraction liquor. When a substantial portion of the wax has been extracted from the mud 11, the solids can be removed by filtration, centrifugation or other means to provide a solution or dispersion of the intermediate wax 35 in the water-miscible solvent containing varying amounts of water. Although the process presently described is a batch-type extraction, continuous extractions and the like can similarly be utilized. The intermediate wax 35 comprises esters of long chain aliphatic alcohols and fatty acids and can be saponified by a variety of means. Preferred saponification methods involve adding a saponification reagent, typically an inorganic base such as sodium or potassium hydroxide to intermediate wax 35, and heating the resulting mixture. After an initial heating period, a substantial portion of the water-miscible solvent can be removed by distillation; a similar portion of water is added to provide saponification products 50. Saponification products 50 include the HPAA's and the salts of fatty acids (soaps). Saponification products 50 are cooled to substantially precipitate the HPAA's. The resulting solids can be removed by filtration, centrifugation, and the like; washed with fresh water, water-miscible solvent, or a combination thereof; and dried to provide: (a) the HPAA's 65 in solid form suitable for use as nutritional and medicinal uses and an aqueous filtrate containing salts of the fatty acids 60. The fatty acids 70 can be isolated by adding an acid to precipitate the fatty acids; filtering, centrifuging or otherwise isolating the precipitated acids and drying the resulting moist acids. Treating the aqueous solution of the acids salt with carbon and/or washing or digesting the solid acid with the water-miscible solvent, water, or a combination thereof can improve the purity and color of the fatty acids recovered.

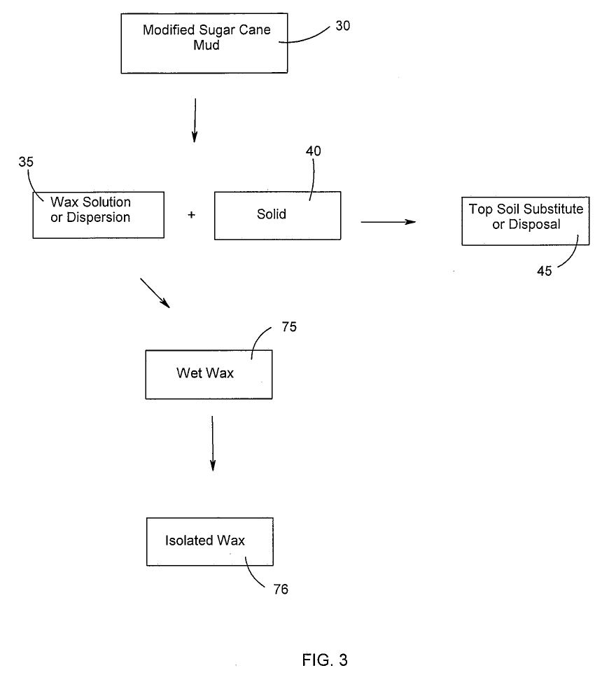

FIG. 3 illustrates the method of processing a modified sugar cane mud or cachaza 30 to obtain a purified form of the wax. Although FIG. 3 provides for a modified mud having a low water content, a raw mud or modified mud having a higher water content can be substituted for modified mud 30. Modified mud 30 is combined with a sufficient amount of water-miscible solvent to extract a substantial portion of the wax present in modified mud 30. Although not required, extraction of wax from the mud is facilitated by heating and agitation of the mixture of mud 30 and the water-miscible solvent. When a substantial portion of the wax present has been extracted, the solid 40 and solution or dispersion 35 are separated. Solution or dispersion 35 is further heated to remove a substantial portion of the water-miscible solvent by distillation and a similar portion of water is added to give an aqueous slurry or dispersion 37 of the wax. Aqueous slurry 37 is cooled to precipitate the wax; the precipitated wax is separated by filtration, centrifugation, and the like; and the wet wax is washed with the water-miscible solvent, water, or a combination thereof; and dried to provide solid wax 75. Color and impurities in the wax can be removed by decolorizing the wax solution or dispersion in the water-miscible solvent and by digesting and/or providing additional washings of the wet wax. Solid 40 can be rinsed with additional water to remove the water-miscible solvent to provide solid 45 that can be disposed of or utilized as a mulch or top soil substitute.

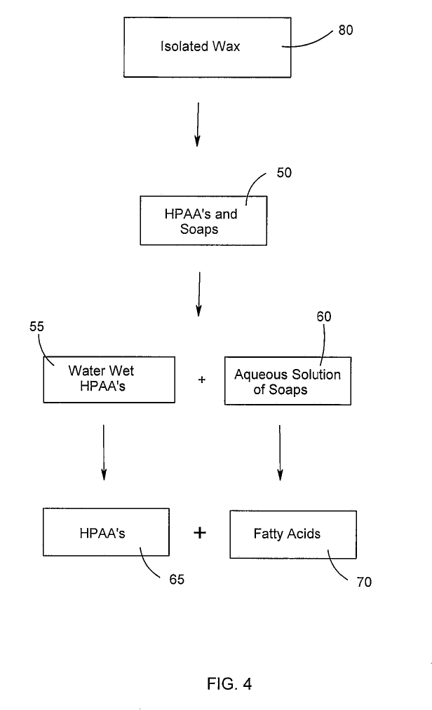

FIG. 4 illustrates the saponification of an isolated solid form of the wax 80. Wax 80 is combined with a water-miscible solvent and a saponification agent to form a saponification mixture and heated to a temperature and for a time sufficient to substantially saponify wax 80. A substantial portion of the water-miscible solvent is removed by distillation, a similar portion of water is added to the saponification mixture, and the resulting saponification mixture cooled to provide an aqueous slurry 50 containing solid HPAA's and the dissolved salts of fatty acids (soaps). Aqueous slurry 50 is filtered to provide an aqueous filtrate 60 containing the salts of the fatty acids and wet solid 55 which consists of the water wet HPAA's. The wet solid 55 can be washed with the water-miscible solvent, water, or mixtures thereof and dried to provide solid HPAA's 65 in a form suitable for nutritional and medicinal uses. Acid can be added to the filtrate 60 to protonate and precipitate the fatty acids 70. The fatty acids 70 can be isolated by filtering the precipitated acids, washing the resulting solid with the water-miscible solvent, water, or mixtures thereof, and drying the wet solid to provide the fatty acids 70 in a form suitable for nutritional and medicinal uses. The color and purity of the long chain aliphatic alcohols and the fatty acids can be further improved by decolorizing solutions of the alcohols and acids before they are precipitated, by digesting and/or providing additional washings with the water-miscible solvent, water, or mixtures thereof.

Although the present disclosure has been provided with reference to specific and preferred parameters, it is apparent that modification and variations thereof may be made by those skilled in the art without departing from the spirit and scope of the present disclosure. The present disclosure is therefore to be considered in all respects as illustrative and not restrictive. In addition, all publications cited herein are indicative of the level of skill in the art and are hereby incorporated by reference in their entirety as if each had been individually incorporated and fully set forth.

I claim:

1. A method for obtaining higher primary aliphatic C₂₄-C₃₆ alcohols (HPAA's) from filter cake mud, the method comprising:
(a) selecting a filter cake mud comprising a wax and a solid fiber, wherein the mud has a water content of up to about 90% water;
(b) extracting the filter cake mud with a water miscible organic solvent to form a mixture containing the solid fiber and the wax dissolved in the water miscible solvent;
(c) clarifying the mixture to provide a solid including fiber and a solution of the wax;
(d) combining the solution with water and a saponification reagent to form a saponification medium;
(e) saponifying the wax in the saponification medium to produce HPAA's;
(f) isolating the HPAA's from the saponification medium.

2. The method of claim 1, wherein the filter cake mud selected was derived from a sugar cane process.

3. The method of claim 2, wherein the filter cake mud selected is a treated filter cake mud derived from an untreated filter cake mud by the method comprising:
(a) mixing the filter cake mud with water to form a first slurry containing water soluble components and water insoluble components;
(b) classifying the first slurry by separating at least a portion of the water insoluble components unable to pass through an opening having a size ranging from about 14 to about 22 mesh to form a second slurry, the second slurry similarly containing water soluble and water insoluble components;
(c) isolating the water insoluble components from the second slurry to form a wet solid; and
(d) drying the wet solid to a water content of from about 20% to about 40% water to form a treated filter cake mud.

4. The method of claim 3, wherein drying involves exposing the isolated water insoluble components to the sun.

5. The method of claim 3, further comprising grinding the treated mud.

6. The method of claim 3, wherein extracting with a water miscible solvent involves extracting with a lower aliphatic alcohol.

7. The method of claim 6, wherein the extracting and clarifying are carried out at an elevated temperature.

8. The method of claim 7, wherein the elevated temperature is at least about 60° C.

9. The method of claim 7, further comprising cooling the clarified solution of wax; precipitating the wax therefrom to provide a slurry containing wax and a solution of alcohol soluble components, and separating the precipitated wax from the slurry to provide a alcohol wet wax.

10. The method of claim 9, further comprising washing the alcohol wet wax with fresh alcohol to provide an alcohol wet wax.

11. The method of claim 7, wherein saponifying includes distilling alcohol from the saponification mixture and replacing the alcohol with a substantially equal volume of water.

12. The method of claim 1, further comprising washing the solid containing fiber with water to provide a substantially solvent free solid and using the substantially solvent free solid as a soil additive or soil substitute.

13. The method of claim 9, wherein separating the alcohol soluble components provides an alcohol solution including oils having a phytosterol component and the method additionally comprises concentrating the oils to provide an oil concentrate and extracting the phytosterol component from the oil concentrate.

14. The method of claim 9, wherein combining includes combining the alcohol wet wax, water and an inorganic base to form the saponification medium.

15. The method of claim 14, wherein combining includes combining an inorganic base selected from the group consisting of sodium hydroxide, potassium hydroxide, and a combination thereof.

16. The method of claim 15, wherein the step of saponifying takes place at an elevated temperature.

17. The method of claim 16, wherein saponifying further includes the steps of removing alcohol and adding water.

18. The method of claim 17, wherein the step of saponifying is carried out at an elevated temperature of at least about 60° C.

19. The method of claim 18, wherein the step of extracting with a lower aliphatic alcohol involves extracting with ethanol and saponifying produces a slurry containing a water-soluble product and a water-insoluble product, wherein the water-insoluble product includes the HPAA's.

20. The method of claim 19, wherein isolating the HPAA's includes filtering the slurry to provide a wet solid and a filtrate; washing the wet solid with ethanol, and drying the wet solid.

21. The method of claim 19, wherein isolating the HPAA's includes centrifuging the slurry to provide a wet solid and a filtrate; washing the wet solid with ethanol, and drying the wet solid.

22. The method of claim 19, wherein isolating the HPAA's includes filtering the slurry to provide a wet solid and a filtrate; washing the wet solid with hot water, and drying the wet solid.

23. The method of claim 19, wherein isolating the HPAA's includes centrifuging the slurry to provide a wet solid and a filtrate; washing the wet solid with hot water, and drying the wet solid.

24. The method of claim 19, wherein saponifying provides the water soluble product comprising salts of long chain fatty acids.

25. The method of claim 20, further comprising the steps of adding a mineral acid to the filtrate to protonate and precipitate the salts of the long chain fatty acids and isolating the long chain fatty acids.

26. A method for obtaining a wax from a filter cake mud comprising:
(a) selecting a filter cake mud having a water content of up to about 90% water;
(b) extracting the filter cake mud with a water miscible organic solvent to form a mixture containing the solid fiber and the wax dissolved in the water miscible solvent;
(c) clarifying the mixture to provide a solution of the wax in the water miscible solvent and the solid fiber;
(d) solidifying the wax to form a slurry; and
(e) isolating the wax from the slurry.

27. The method of claim 26, wherein isolating the wax further includes washing the wax.

28. The method of claim 26, wherein isolating the wax further includes drying the wax.

29. The method of claim 26, wherein extracting the filter cake mud with a water miscible organic solvent includes extracting the mud with a lower aliphatic alcohol.

30. The method of claim 29, wherein extracting the filter cake mud with a lower aliphatic alcohol includes extracting the mud with ethanol.

31. The method of claim 26, wherein selecting a filter cake mud includes selecting a filter cake mud having a water content of from about 20% to about 40% water,

32. A method for saponifying a wax containing esters of higher primary aliphatic C₂₄-C₃₆ alcohols (HPAA's) and fatty acids comprising:
(a) forming a saponification medium containing the wax, a water-miscible solvent, and a saponification reagent;
(b) saponifying the wax in the saponification medium to produce long chain aliphatic alcohols and fatty acids; and
(c) isolating the HPAA's from the saponification medium.

33. The method of claim 32, wherein forming a saponification medium involves a water miscible solvent comprising a lower aliphatic alcohol.

34. The method of claim 33, wherein forming a saponification medium involves a water miscible solvent comprising ethanol.

35. The method of claim 32, wherein forming a saponification medium involves a saponification reagent comprising an inorganic base.

36. The method of claim 35, wherein forming a saponification medium involves a saponification reagent comprising an inorganic base selected from the group consisting of sodium hydroxide, potassium hydroxide, and combinations thereof.

37. The method of claim 32, wherein isolating the HPAA's includes filtering the HPAA's to provide a wet solid and a filtrate; washing the wet solid with the water miscible solvent, and drying the wet solid.

38. The method of claim 32, wherein isolating the HPAA's includes filtering the HPAA's to provide a wet solid and a filtrate; washing the wet solid with water, and drying the wet solid.

39. The method of claim 32, wherein isolating the HPAA's includes centrifuging the HPAA's to provide a wet solid and a filtrate; washing the wet solid with the water miscible solvent, and drying the wet solid.

40. The method of claim 32, wherein isolating the HPAA's includes centrifuging the HPAA's to provide a wet solid and a filtrate; washing the wet solid with water, and drying the wet solid.

41. The method of claim 32, further comprising isolating the fatty acids.

42. The method of claim 37, further comprising the steps of adding a mineral acid to the filtrate to protonate and precipitate the salts of the long chain fatty acids and isolating the long chain fatty acids.

43. A method for forming a modified filter cake mud comprising:
(a) selecting a raw filter cake mud containing water insoluble particles having a range of sizes;
(b) forming a slurry containing the filter cake mud and water;
(c) classifying the slurry by separating water insoluble particles capable of passing through an opening of from about 14 to about 22 mesh to form a second slurry; and
(d) isolating water insoluble components from the second slurry to provide a water wet solid.

44. The method of claim 43, further comprising drying the water wet solid by exposing the water wet solid to the sun.

45. The method of claim 43, further comprising drying the water wet solid to a water content of less than about 70%.

46. The method of claim 43, further comprising drying the water wet solid to a water content of less than about 40%.

47. The method of claim 43, further comprising drying the water wet solid to a water content of about 20% to provide a modified mud that has a reduced volume and that can resist biological degradation.
